(19) United States
(12) Patent Application Publication
(10) Pub. No.: US 2004/0248865 A1
(43) Pub. Date: Dec. 9, 2004

(54) Antiarthritic combinations

(75) Inventors: Heather A. Harris, Phoenixville, PA (US); Damien P. Quinn, Wayne, PA (US); Regina H. Kurrasch, King of Prussia, PA (US)

Correspondence Address: COZEN O' CONNOR, P. C., 1900 MARKET STREET, PHILADELPHIA, PA, 19103-3508, US

(73) Assignee: Wyeth, Madison, NJ

(21) Appl. No.: 10/849,761

(22) Filed: May 20, 2004

Related U.S. Application Data
(60) Provisional application No. 60/472,382, filed on May 21, 2003.

(51) Int. Cl. A61K 31/56

(52) U.S. Cl. 514/171

(57) ABSTRACT

This invention provides combinations of an ERβ selective ligand with an anti-arthritis agent, which are useful in the treatment or inhibition of arthritis.

### CROSS REFERENCE TO RELATED APPLICATIONS

This application claims priority benefit of U.S. Provisional Application Ser. No. 60/472,382, filed May 21, 2003, the entire content of which is incorporated by reference herein.

### BACKGROUND OF THE INVENTION

This invention relates to combinations of an ERβ selective ligand with an anti-arthritis agent, which are useful in the treatment or inhibition of arthritis.

Arthritis is a broad term encompassing three diseases: rheumatoid arthritis, osteoarthritis, and spondyloarthropathies. Characteristics of these disorders are more fully described below

Rheumatoid arthritis: In this disease, the synovial tissue lining the joint organizes into a mass that infiltrates and degrades articular cartilage, tendons and bone. Normal synovial tissue consists of a thin membrane of only two or three cell layers, comprised principally of fibroblast-like synovial cells and rare resident macrophages. In contrast, rheumatoid synovial tissue consists of a mixture of cell types: immune T- and B-cells, monocytes/macrophages, polymorphonuclear leukocytes and the fibroblast-like cells with their rampant proliferative ability. With the exception of the fibroblasts, most of these cells are recruited to the rheumatoid joint in response to inflammatory stimuli that occur as part of the pathology of this disease.

Although the etiology of rheumatoid arthritis is not clear, it is suspected that an unknown antigen (such as a bacterium, virus or mycoplasma) is deposited in the joints as a consequence of a systemic infection. Normally the antigen is cleared and no disease arises. However, in certain individuals, the antigen elicits an acute inflammatory/foreign body response in which some autologous tissue damage occurs. This, in turn, develops into an autoimmune response and eventually leads to a chronic inflammatory and immunologic reaction within the synovial lining of the joint. Thus there is a potpourri of activated cell types, and the cytokines they produce continuously fuel the proliferative and destructive ability of the synovial fibroblasts.

Osteoarthritis: In osteoarthritis, degenerative changes to the articular cartilage, subchondral bone and the synovial membrane occur after various joints are subjected to repeated mechanical damage. Increased levels of IL-1, TNF-α and metalloproteases have been demonstrated within the joints of affected individuals.

Spondyloarthropathies: The diseases classified as spondyloarthropathies are psoriatic arthritis, juvenile chronic arthritis with late pannus onset, enterogenic spondyloarthropathies (enterogenic reactive arthritis and inflammatory bowel diseases), urogenital spondyloarthropathies, and undifferentiated spondyloarthropathies.

In this type of arthridity, various types of immune-mediated joint inflammation produce degenerative changes in the multiple joints. These changes consist of inflammatory infiltration within the synovial membranes and degenerative changes to articular cartilage and the associated subchondral bone. One additional feature of this particular syndrome is the development of bony bridges (spondyloses) between affected joint components. Again, increased levels of IL-1, TNF-α and metalloproteases have been documented within affected joints of patients.

Standard medical therapy for arthritis includes oral preparations of steroids (i.e. corticosteroids) and nonsteroidal antiinflammatory agents (e.g. naproxen and celecoxib) to reduce inflammation and control pain. More recently, INFLIXIMAB, a chimeric monoclonal antibody to tumor necrosis factor-alpha and etanercept, a fusion protein (pseudoreceptor) that prevents tumor necrosis factor from binding its receptor, have been approved for the treatment of rheumatoid arthritis. With each of these therapies however, the efficacy, method of delivery and side effect profile are still major concerns.

A well-established model of arthritis is the Lewis rat model of adjuvant-induced arthritis, where severe joint swelling occurs [Kahan, et al., Agents & Actions 6: 219 (1976), Leisten, et al., Clinical Immunology & Immunopathology 56: 108 (1990);]. A second model is the HLA-B27 rat which exhibits a spontaneous inflammatory disease phenotype [Taurog, et al., Journal of Immunology 150: 4168 (1993);] resulting in joint swelling.

Estrogens have previously been shown to modulate the immune system both though direct effect on immune system cells, e.g. cell survival, (for review see Cutolo, et al., Clinical & Experimental Rheumatology 13: 217 (1995), Jansson and Holmdahl, Inflammation Research 47: 290 (1998);) and though influencing cytokine production via NF-κB inhibition [Ray, et al., Journal of Biological Chemistry 269: 12940 (1994), Stein and Yang, Molecular & Cellular Biology 15: 4971 (1995);]. Estrogens exert their actions in cells by binding to receptors, of which two are known. The second form of the estrogen receptor (ER) was recently discovered [Tremblay, et al., Molecular Endocrinology 11: 353 (1997), Bhat, et al., Journal of Steroid Biochemistry & Molecular Biology 67: 233 (1998), Kuiper, et al., Proceedings of the National Academy of Sciences of the United States of America 93: 5925 (1996), Mosselman, et al., FEBS Letters 392: 49 (1996);]. This protein has been designated ERβ to distinguish it from the previously known form, now called ERα. Although the tissue distribution [Brandenberger, et al., Journal of Clinical Endocrinology & Metabolism 82: 3509 (1997), Kuiper, et al., Endocrinology 138: 863 (1997), Saji, et al., Proceedings of the National Academy of Sciences of the United States of America 97: 337 (2000), Saunders, et al., Journal of Endocrinology 154: R13 (1997), Shughrue, et al., Journal of Comparative Neurology 388: 507 (1997), Taylor and Al-Azzawi, Journal of Molecular Endocrinology 24: 145 (2000), Vidal, et al., Journal of Bone & Mineral Research 14: 923 (1999), Wang, et al., Biology of Reproduction 63: 1331 (2000);] ligand specificity [Kuiper, et al., Endocrinology 138: 863 (1997), Kuiper, et al., Endocrinology 139: 4252 (1998);] and three-dimensional structure (through X-ray crystallography) [Pike, et al., EMBO Journal 18: 4608 (1999);] of ERβ have been studied, defining its function has remained elusive.

Of interest to this invention, is the observation that ERβ can interfere with NF-κB transcriptional activation, as evaluated using the non-receptor selective estrogen, 17β-estradiol [Bhat, et al., Journal of Steroid Biochemistry & Molecular Biology 67: 233 (1998), Harnish, et al., Endocrinology 141: 3403 (2000);]. In addition, ERβ is expressed in growth plate and articular cartilage [Juma, et al., Journal of Bone & Mineral Research 14: (1999), Nilsson, et al., Journal of Clinical Endocrinology & Metabolism 84: 370 (1999), Nilsson, et al., Hormone Research 51: 23 (1999), Nilsson, et al., Journal of Bone & Mineral Research 14: (1999), Savendahl, et al., Acta Paediatrica. Supplement (2000, Ushiyama, et al., Osteoarthritis & Cartilage 7: 560 (1999, Richmond, et al., Arthritis & Rheumatism 43: 2081 (2000);], and cells of the immune system [Mosselman, et al., FEBS Letters 392: 49 (1996), Brandenberger, et al., Journal of Clinical Endocrinology & Metabolism 82: 3509 (1997), Rider, et al., Clinical Immunology 95: 124 (2000);]. Additionally, it has recently been shown that ICI-182780 (a nonselective estrogen receptor antagonist) can enhance development of arthritis in normal cycling female mice [Jansson, L., Arthritis & Rheumatism 44: 2168 (2001);].

### SUMMARY OF THE INVENTION

In some embodiments, the present invention provides methods of treating or inhibiting arthritis in a mammal in need thereof, which comprises providing to said mammal an effective amount of a combination of a non-uterotrophic, non-mammotrophic ERβ selective ligand, wherein the binding affinity of the ER-β selective ligand to ER-β is at least about 20 times greater than its binding affinity to ER-α, and an anti-arthritis agent.

In some embodiments, the binding affinity of the ERβ selective ligand to ERβ is at least about 50 times greater than its binding affinity to ERα. In some such embodiments, the ERβ selective ligand causes an increase in wet uterine weight which is less than about 10% of that observed for a maximally efficacious dose of 17β-estradiol in a standard pharmacological test procedure measuring uterotrophic activity, and the ERβ selective ligand has activity that is <10% as efficacious as 17beta-estradiol at facilitating the development of lobular-alveolar end buds as assessed by histological examination.

In some of the foregoing embodiments, the ERβ selective ligand does not significantly Increase wet uterine weight compared with a control that is devoid of uterotrophic activity, and does not significantly facilitate the development of lobular-alveolar end buds compared with a control that is devoid of mammotrophic activity.

In some embodiments, the present invention provides methods of treating or inhibiting joint swelling or erosion; or treating or inhibiting joint damage secondary to arthroscopic or surgical procedures in a mammal in need thereof, which comprises providing to said mammal an effective amount of a combination of a non-uterotrophic, non-mammotrophic ERβ selective ligand, wherein the binding affinity of the ER-β selective ligand to ER-β is at least about 20 times greater than its binding affinity to ER-α, and an anti-arthritis agent. In some embodiments, the binding affinity of the ERβ selective ligand to ERβ is at least about 50 times greater than its binding affinity to ERα.

In some further embodiments, the invention provides methods of treating or inhibiting arthritis in a mammal in need thereof, which comprises providing to said mammal an effective amount of a combination of a compound of formula I, having the structure:

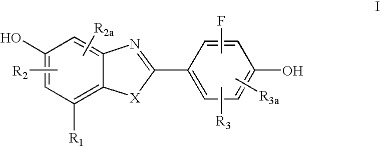

wherein

R₁ is alkenyl of 2-7 carbon atoms; wherein the alkenyl moiety is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₅, R₆ are each, independently hydrogen, alkyl of 1-6 carbon atoms, aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅;

or a pharmaceutically acceptable salt thereof, and an anti-arthritis agent. In some embodiments, X is O. In further embodiments, R₁ is alkenyl of 2-3 carbon atoms, which is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆. In some embodiments, the compound of formula I is 2-(3-fluoro-4-hydroxyphenyl)-7-vinyl-1,3-benzoxazol-5-ol or a pharmaceutically acceptable salt thereof.

Also provided in accordance with the present invention are methods of treating or inhibiting joint swelling or erosion; or treating or inhibiting joint damage secondary to arthroscopic or surgical procedures in a mammal in need thereof, which comprises providing to said mammal an effective amount of a combination of 2-(3-fluoro-4-hydroxyphenyl)-7-vinyl-1,3-benzoxazol-5-ol or a pharmaceutically acceptable salt thereof, and an anti-arthritis agent.

The invention further provides methods of treating or inhibiting arthritis in a mammal in need thereof, which comprises providing to said mammal an effective amount of a combination of a compound of formula II, having the structure:

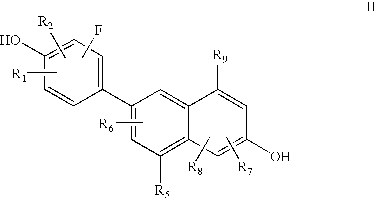

wherein

R₁ and R₂ are each, independently, selected from hydrogen, hydroxyl, alkyl of 1-6 carbon atoms, alkenyl, of 2-7 carbon atoms, and alkynyl of 2-7 carbon atoms, alkoxy of 1-6 carbon atoms, or halogen;

R₅, R₆, R₇, R₈, and R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluroalkyl, trifluoroalkoxy, —NO₂, or phenyl; wherein the phenyl moiety of R₅, R₆, R₇, R₈, R₉, or R₁₀ may be optionally mono-, di-, or tri-subsitituted with alkyl of 1-6 carbon atoms, alkenyl of 2-7 carbon atoms, halogen, hydroxyl, alkoxy of 1-6 carbon atoms, halogen, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

with the proviso that at least one of R₅ or R₉ is not hydrogen; or a pharmaceutically acceptable salt thereof. In some embodiments, the compound of formula II has the structure:

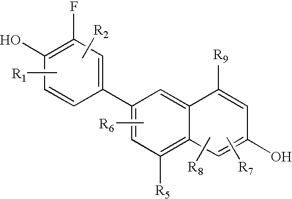

or a pharmaceutically acceptable salt thereof. In some such embodiments, the 5 or 6-membered heterocyclic ring having 1 to 4 heteroatoms selected from O, N or S is furan, thiophene, or pyridine or a pharmaceutically acceptable salt thereof. In further embodiments, R₅, R₆, R₇, R₈, and R₉ are each, independently, hydrogen, halogen, —CN, or alkynyl of 2-7 carbon atoms. In some further embodiments, R₆, R₇, and R₈ are hydrogen.

In some embodiments, the compound of formula II is 3-(3-fluoro-4-hydroxyphenyl)-7-hydroxy-1-naphthonitrile or a pharmaceutically acceptable salt thereof.

The present invention further provides methods of treating or inhibiting joint swelling or erosion; or treating or inhibiting joint damage secondary to arthroscopic or surgical procedures in a mammal in need thereof, which comprises providing to said mammal an effective amount of a combination of 3-(3-fluoro-4-hydroxyphenyl)-7-hydroxy-1-naphthonitrile or a pharmaceutically acceptable salt thereof, and an anti-arthritis agent.

In some of the foregoing embodiments, the arthritis is rheumatoid arthritis, osteoarthritis, or spondyloarthropathies. In some of the foregoing embodiments, the anti-arthritis agent is useful in treating the signs and symptoms of arthritis or is a disease modifying antirheumatic drug. In some of the foregoing embodiments, the anti-arthritis agent is a) a steroidal antiinflammatory agent, b) sulfasalazine, c) methotrexate, d) auranofin, e) D-penicillamine, f) a COX-2 inhibitor, g) an NSAID, i) a P38 MAP kinase inhibitor, j) a TNFα inhibitor, k) a IL1β converting enzyme inhibitor, l) a VLA4 antagonist, m) a NFκB inhibitor, n) an immunomodulator, o) a IL1 receptor antagonist, p) an antibiotic, q) a statin, r) cyclophosphamide, s) hydroxychloroquine, or t) chlorambusil.

### DESCRIPTION OF THE INVENTION

This invention provides a combination of an ERβ selective ligand plus an anti-arthritis agent for the use in treating or inhibiting arthritis. More particularly, the combination is useful in the treatment or inhibition of rheumatoid arthritis, osteoarthritis or spondyloarthropathies, or the treatment or inhibition of joint swelling or erosion; or treating or inhibiting joint damage secondary to arthroscopic or surgical procedures. This invention also provides combinations of an ERβ selective ligand and an anti-arthritis agent in which the dosage of either the ERβ selective ligand or the anti-arthritis agent or both are used in subtherapeutically effective dosages.

As used in accordance with this invention, the term “providing,” with respect to providing a compound or substance covered by this invention, means either directly administering such a compound or substance, or administering a prodrug, derivative, or analog which will form the effective amount of the compound or substance within the body.

As used in accordance with this invention, the term “treatment” means treating a mammal having or being susceptible to arthritis by providing said mammal an effective amount of a ERβ selective ligand with the purpose of inhibiting onset or progression of the arthritis, eradicating arthritis, or palliating the arthritis.

As used in accordance with this invention, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERα and ERβ. Such a standard test is disclosed in, for example, Harris, H. A., et al., Steroids 67 (2002) 379-384, incorporated by reference herein in its entirety. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα. It is further preferred that the ERβ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of 17β-estradiol or 17α-ethinyl-17β-estradiol in the same procedure. Such a standard test is disclosed in, for example, Harris, H. A., et al., Endocrinology 143(11) 4172-4177 (2002), incorporated by reference herein in its entirety. It is preferred that the increase in wet uterine weight will be less than about 25% of that observed for estradiol, and more preferred that the increase in wet uterine weight will be less than about 10% of that observed for estradiol. It is most preferred that the non-uterotrophic ERβ selective ligand will not increase wet uterine weight significantly compared with a control that is devoid of uterotrophic activity (e.g. vehicle). In the context of the present invention, the non-uterotrophic ERβ selective ligand is considered to not increase wet uterine weight significantly compared with such a control of uterotrophic activity if the confidence level of such a comparison is less than 0.05 (i.e., p<0.05).

As used in accordance with this invention, the term “non-mammotrophic” means having activity that is <10% as efficacious as 17beta-estradiol at facilitating the development of lobular-alveolar end buds as assessed by histological examination. Examples of such determination by histological examination are well known in the art. See, for example, Harris, H. A., et al., Endocrinology 144(10) 4241-4249 (2003); Mulac-Jericevic, B., et al., Proc. Natl. Acad. Sci., 100 (17) 9744-9749 (2003); Bocchinfuso, W. P., et al., Endocrinology 141(8) 2982-2994 (2002); and Lewis, B. C., et al., Toxicological Sciences 62, 46-53 (2001), each of which is incorporated by reference herein in its entirety.

As used in accordance with this invention, the term “anti-arthritis agent” means an compound, agent, or substance which is useful in treating or inhibiting the signs or symptoms of arthritis or is a disease modifying antirheumatic drug (DMARD).

Preferred anti-arthritis agents include, but are not limited to the following. These agents are either commercially available, or can be prepared as described further below.

a) steroidal antiinflammatory agents, such as prednisolone, dexamethasone, or cortisone;

b) sulfasalazine;

c) methotrexate;

d) auranofin;

e) D-penicillamine;

f) COX-2 inhibitors, such as celecoxib or rofecoxib;

g) NSAIDs, such as etodolac, ibuprofen, naproxen, or piroxicam;

h) lefunomide;

i) P38 MAP kinase inhibitors, such as SCIO-469 (disclosed in U.S. Pat. No. 6,541,477 and U.S. Pat. No. 6,410,540, which are hereby incorporated by reference);

j) TNFα inhibitors, such as TMI-005 (disclosed in U.S. Pat. No. 6,225,311, which is hereby incorporated by reference), ISIS 104838 (disclosed in U.S. Pat. No. 6,080,580, which is hereby incorporated by reference), infliximab, or entanercept;

k) IL1β converting enzyme inhibitors;

l) VLA4 antagonists;

m) NFκB inhibitors;

n) immunomodulators, such as CCI-779 (U.S. Pat. No. 5,362,718), rapamune, cyclosporine, fludarabine, or leflunomide;

o) IL1 receptor antagonists, such as anakinra;

p) antibiotics, such as minocycline;

q) statins, such as simvastatin;

r) cyclophosphamide;

s) hydroxychloroquine; or

t) chlorambusil

As used herein, the term “Interleukin-1β converting enzyme inhibitors” (also known as caspase-1 inhibitors) is intended to mean agents that inhibit the release of active Interleukin-1β from a proenzyme form.

As used herein, the term “VLA4 antagonists” means agents that block cell signaling mediated by very late antigen 4 (also known as α4β1 integrin or CD49D/CD29). The anti-inflammatory effect of blocking this protein has been described [Lin and Castro, Current Opinion in Chemical Biology 2: 453-457 (1998), incorporated herein by reference in its entirety].

As used herein, the term “Nuclear factor kappa B (NFκB)” has its recognized meaning of a transcription factor that plays a key role in propagating the inflammation cascade.

As used herein, the term “Interleukin-1 (IL-1) receptor antagonists” denotes agents that bind the IL-1 receptor but do not elicit the same proinflammatory response. Their effect at blunting inflammation has been described [Arend et. al. Annual Review of immunology 16:27-55 (1998), incorporated herein by reference in its entirety].

Preferred ERβ selective ligands include those of formula I having the structure:

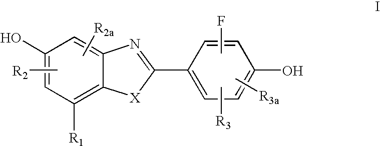

wherein

R₁ is alkenyl of 2-7 carbon atoms; wherein the alkenyl moiety is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;

R₅, R₆ are each, independently hydrogen, alkyl of 1-6 carbon atoms or aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅;

or a pharmaceutically acceptable salt thereof.

The preparation and use of compounds of formula I is described in U.S. patent application Ser. No. 10/309,699, which is hereby incorporated by reference in its entirety.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable aids when a compound of this invention contains a basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when a compound of this invention contains an acidic moiety.

The terms alkyl, alkenyl, and alkynyl include both branched and straight chain moieties. Examples include methyl, ethyl, propyl, butyl, isopropyl, sec-butyl, tert-butyl, vinyl, allyl, acetylene, 1-methyl vinyl, and the like. When alkyl or alkenyl moieties are substituted, they may typically be mono-, di-, tri- or persubstituted. Examples for a halogen substituent include 1-bromo vinyl, 1-fluoro vinyl, 1,2-difluoro vinyl, 2,2-difluorovinyl, 1,2,2-trifluorovinyl, 1,2-dibromo ethane, 1,2 difluoro ethane, 1-fluoro-2-bromo ethane, CF₂CF₃, CF₂CF₂CF₃, and the like. The term halogen includes bromine, chlorine, fluorine, and iodine. The term aryl means phenyl, 1-naphthyl, or 2-naphthyl. Preferred 5-6 membered heterocyclic rings include furan, thiophene, pyrrole, isopyrrole, pyrazole, imidazole, triazole, dithiole, oxathiole, isoxazole, oxazole, thiazole, isothiazolem oxadiazole, furazan, oxatriazole, dioxazole, oxathiazole, tetrazole, pyran, pyridine, pyridazine, pyrimidine, pyrazine, triazine, oxazine, oxathiazine, or oxadiazine. It is more preferred that the heterocyclic ring is furan, thiophene, or thiazole.

Of the compounds of formula I, it is preferred that X is O; and that X is O and R₁ is alkenyl of 2-3 carbon atoms, which is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆. In one particularly preferred embodiment, the compound of formula I is 2-(3-fluoro-4-hydroxyphenyl)-7-vinyl-1,3-benzoxazol-5-ol or a pharmaceutically acceptable salt thereof.

Other preferred ERβ selective ligands are those of formula II, having the structure:

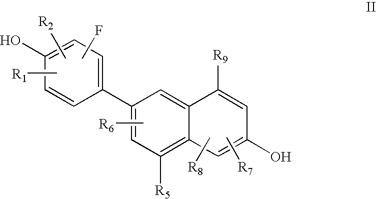

wherein

R₁ and R₂ are each, independently, hydrogen, hydroxyl, alkyl of 1-6 carbon atoms, alkenyl of 2-7 carbon atoms, alkynyl of 2-7 carbon atoms, alkoxy of 1-6 carbon atoms or halogen;

R₅, R₆, R₇, R₈, and R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluroalkyl, trifluoroalkoxy, —NO₂, or phenyl; wherein the phenyl moiety of R₅, R₆, R₇, R₈, R₉, or R₁₀ may be optionally mono-, di-, or tri-subsitituted with up to three substituents independently selected from alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

with the proviso that at least one of R₅ or R₉ is not hydrogen;

or a pharmaceutically acceptable salt thereof.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable aids when a compound of this invention contains a basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when a compound of this invention contains an acidic moiety.

The terms alkyl and alkenyl include both branched and straight chain moieties. Examples include methyl, ethyl, propyl, butyl, isopropyl, sec-butyl, tert-butyl, vinyl, allyl, 1-methyl vinyl, and the like. When alkyl or alkenyl moieties are substituted, they may typically be mono-, di-, tri- or persubstituted. Examples for a halogen substituent include 1-bromo vinyl, 1-fluoro vinyl, 1,2-difluoro vinyl, 2,2-difluorovinyl, 1,2,2-trifluorovinyl, 1,2-dibromo ethane, 1,2 difluoro ethane, 1-fluoro-2-bromo ethane, CF₂CF₃, CF₂CF₂CF₃, and the like. The term halogen includes bromine, chlorine, fluorine, and iodine.

Preferred 5-6 membered heterocyclic rings include furan, thiophene, pyrrole, isopyrrole, pyrazole, imidazole, triazole, dithiole, oxathiole, isoxazole, oxazole, thiazole, isothiazolem oxadiazole, furazan, oxatriazole, dioxazole, oxathiazole, tetrazole, pyran, pyridine, pyridazine, pyrimidine, pyrazine, triazine, oxazine, oxathiazine, or oxadiazine. It is more preferred that the heterocyclic ring is furan, thiophene, or pyridine.

The preparation and use of compounds of formula II is described in U.S. patent application Ser. No. 10/316,640, which is hereby incorporated by reference.

Preferred compounds of formula II are those having the structure:

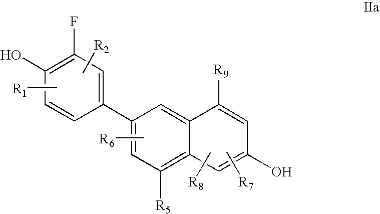

or a pharmaceutically acceptable salt thereof.

More preferred compounds of formula II, are those having the structure of IIa, and:

wherein the 5 or 6-membered heterocyclic ring having 1 to 4 heteroatoms selected from O, N or S is furan, thiophene, or pyridine or a pharmaceutically acceptable salt thereof.

More preferred compounds of formula IIa include those in which the 5 or 6-membered heterocyclic ring having 1 to 4 heteroatoms selected from O, N or S is furan, thiophene, or pyridine; and R₅, R₆, R₇, R₈, and R₉ are each, independently, hydrogen, halogen, —CN, or alkynyl of 2-7 carbon atoms; or a pharmaceutically acceptable salt thereof.

More preferred compounds of formula IIa also include those in which the 5 or 6-membered heterocyclic ring having 1 to 4 heteroatoms selected from O, N or S is furan, thiophene, or pyridine; R₅, and R₉ are each, independently, hydrogen, halogen, —CN, or alkynyl of 2-7 carbon atoms; and R₆, R₇, and R₈ are hydrogen; or a pharmaceutically acceptable salt thereof.

In one particularly preferred embodiment, the compound of formula II is 3-(3-fluoro-4-hydroxyphenyl)-7-hydroxy-1-naphthonitrile or a pharmaceutically acceptable salt thereof.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the ERβ selective ligand may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration. Dosages of the anti-arthritis agents will vary by the selection of the agent and route of administration. In general, the initial dosage will be the commercially available dosage and route of administration, for those agents that are commercially available. Agents not commercially available will be administered in accordance with dosages and routes of administration described in the literature.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

As those skilled in the art will appreciate, numerous changes and modifications may be made to the preferred embodiments of the invention without departing from the spirit of the invention. It is intended that all such variations fall within the scope of the invention.

It is intended that each of the patents, applications, and printed publications including books mentioned in this patent document be hereby incorporated by reference in their entirety.

What is claimed is:

1. A method of treating or inhibiting arthritis in a mammal in need thereof, which comprises providing to said mammal an effective amount of a combination of a non-uterotrophic, non-mammotrophic ERβ selective ligand, wherein the binding affinity of the ER-β selective ligand to ER-β is at least about 20 times greater than its binding affinity to ER-α, and an anti-arthritis agent.

2. The method according to claim 1, wherein the arthritis is rheumatoid arthritis, osteoarthritis, or spondyloarthropathies.

3. The method according to claim 2, wherein the binding affinity of the ERβ selective ligand to ERβ is at least about 50 times greater than its binding affinity to ERα.

4. The method according to claim 3, wherein the ERβ selective ligand causes an increase in wet uterine weight which is less than about 10% of that observed for a maximally efficacious dose of 17β-estradiol, and the ERβ selective ligand has activity that is <10% as efficacious as 17beta-estradiol at facilitating the development of lobular-alveolar end buds as assessed by histological examination.

5. The method according to claim 4, wherein the ERβ selective ligand does not significantly increase wet uterine weight compared with a control that is devoid of uterotrophic activity, and does not significantly facilitate the development of lobular-alveolar end buds compared with a control that is devoid of mammotrophic activity.

6. The method according to claim 1, wherein the anti-arthritis agent is useful in treating the signs and symptoms of arthritis or is a disease modifying antirheumatic drug.

7. The method according to claim 6, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic,
q) a statin,
r) cyclophosphamide,
s) hydroxychloroquine, or
t) chlorambusil.

8. The method according to claim 5, wherein the anti-arthritis agent is useful in treating the signs and symptoms of arthritis or is a disease modifying antirheumatic drug.

9. The method according to claim 8, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

10. A method of treating or inhibiting joint swelling or erosion; or treating or inhibiting joint damage secondary to arthroscopic or surgical procedures in a mammal in need thereof, which comprises providing to said mammal an effective amount of a combination of a non-uterotrophic, non-mammotrophic ERβ selective ligand, wherein the binding affinity of the ER-β selective ligand to ER-β is at least about 20 times greater than its binding affinity to ER-α, and an anti-arthritis agent.

11. The method according to claim 10, wherein the binding affinity of the ERβ selective ligand to ERβ is at least about 50 times greater than its binding affinity to ERα.

12. The method according to claim 11, wherein the anti-arthritis agent is useful in treating the signs and symptoms of arthritis or is a disease modifying antirheumatic drug.

13. The method according to claim 12, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

14. A method of treating or inhibiting arthritis in a mammal in need thereof, which comprises providing to said mammal an effective amount of a combination of a compound of formula I, having the structure 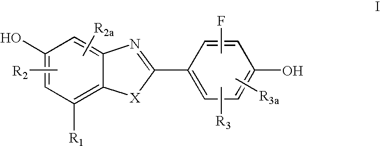
wherein
R₁ is alkenyl of 2-7 carbon atoms; wherein the alkenyl moiety is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof, and an anti-arthritis agent.

15. The method according to claim 14, wherein X is O.

16. The method according to claim 15, wherein R₁ is alkenyl of 2-3 carbon atoms, which is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆.

17. The method according to claim 14, the compound of formula I is 2-(3-fluoro-4-hydroxyphenyl)-7-vinyl-1,3-benzoxazol-5-ol or a pharmaceutically acceptable salt thereof.

18. The method according to claim 14, wherein the arthritis is rheumatoid arthritis, osteoarthritis, or spondyloarthropathies.

19. The method according to claim 17, wherein the arthritis is rheumatoid arthritis, osteoarthritis, or spondyloarthropathies.

20. The method according to claim 14, wherein the anti-arthritis agent is useful in treating the signs and symptoms of arthritis or is a disease mofifying anthrheumatic drug.

21. The method according to claim 20, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

22. The method according to claim 17, wherein the anti-arthritis agent is useful in treating the signs and symptoms of arthritis or is a disease mofifying anthrheumatic drug.

23. The method according to claim 22, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

24. A method of treating or inhibiting joint swelling or erosion; or treating or inhibiting joint damage secondary to arthroscopic or surgical procedures in a mammal in need thereof, which comprises providing to said mammal an effective amount of a combination of 2-(3-fluoro-4-hydroxyphenyl)-7-vinyl-1,3-benzoxazol-5-ol or a pharmaceutically acceptable salt thereof, and an anti-arthritis agent.

25. A method of treating or inhibiting arthritis in a mammal in need thereof, which comprises providing to said mammal an effective amount of a combination of a compound of formula II, having the structure 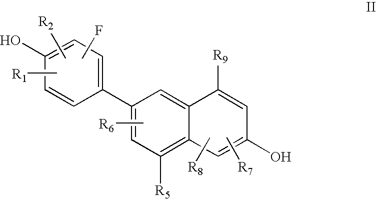
wherein
R₁ and R₂ are each, independently, selected from hydrogen, hydroxyl, alkyl of 1-6 carbon atoms, alkenyl, of 2-7 carbon atoms, and alkynyl of 2-7 carbon atoms, alkoxy of 1-6 carbon atoms, or halogen;
R₅, R₆, R₇, R₈, and R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluroalkyl, trifluoroalkoxy, —NO₂, or phenyl; wherein the phenyl moiety of R₅, R₆, R₇, R₈, R₉, or R₁₀ may be optionally mono-, di-, or tri-subsitituted with alkyl of 1-6 carbon atoms, alkenyl of 2-7 carbon atoms, halogen, hydroxyl, alkoxy of 1-6 carbon atoms, halogen, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
with the proviso that at least one of R₅ or R₉ is not hydrogen, or a pharmaceutically acceptable salt thereof.

26. The method according to claim 25, wherein the compound of formula II has the structure 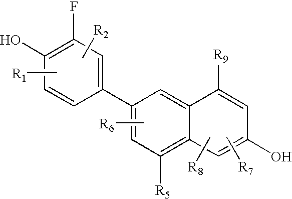
or a pharmaceutically acceptable salt thereof.

27. The method according to claim 26 wherein the 5 or 6-membered heterocyclic ring having 1 to 4 heteroatoms selected from O, N or S is furan, thiophene, or pyridine or a pharmaceutically acceptable salt thereof.

28. The method according to claim 27, wherein R₅, R₆, R₇, R₈, and R₉ are each, independently, hydrogen, halogen, —CN, or alkynyl of 2-7 carbon atoms or a pharmaceutically acceptable salt thereof.

29. The method according to claim 28, wherein R₆, R₇, and R₈ are hydrogen, or a pharmaceutically acceptable salt thereof.

30. The method according to claim 25, wherein the compound of formula II is 3-(3-fluoro-4-hydroxyphenyl)-7-hydroxy-1-naphthonitrile or a pharmaceutically acceptable salt thereof.

31. The method according to claim 25, wherein the arthritis is rheumatoid arthritis, osteoarthritis, or spondyloarthropathies.

32. The method according to claim 30, wherein the arthritis is rheumatoid arthritis, osteoarthritis, or spondyloarthropathies.

33. The method according to claim 25, wherein the anti-arthritis agent is useful in treating the signs and symptoms of arthritis or is a disease mofifying anthrheumatic drug.

34. The method according to claim 33, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

35. The method according to claim 30, wherein the anti-arthritis agent is useful in treating the signs and symptoms of arthritis or is a disease mofifying anthrheumatic drug.

36. The method according to claim 35, wherein the anti-arthritis agent is:
a) a steroidal antiinflammatory agent,
b) sulfasalazine,
c) methotrexate,
d) auranofin,
e) D-penicillamine,
f) a COX-2 inhibitor,
g) an NSAID,
i) a P38 MAP kinase inhibitor,
j) a TNFα inhibitor,
k) a IL1β converting enzyme inhibitor,
l) a VLA4 antagonist,
m) a NFκB inhibitor,
n) an immunomodulator,
o) a IL1 receptor antagonist,
p) an antibiotic, or
q) a statin,
r) cyclophosphamide,
s) hydroxychloroquine, or
t) chlorambusil.

37. A method of treating or inhibiting joint swelling or erosion; or treating or inhibiting joint damage secondary to arthroscopic or surgical procedures in a mammal in need thereof, which comprises providing to said mammal an effective amount of a combination of 3-(3-fluoro-4-hydroxyphenyl)-7-hydroxy-1-naphthonitrile or a pharmaceutically acceptable salt thereof, and an anti-arthritis agent.
